(19) United States
(12) Patent Application Publication
(10) Pub. No.: US 2003/0191396 A1
(43) Pub. Date: Oct. 9, 2003

(54) Tissue treatment method and apparatus

(76) Inventors: Narendra T Sanghvi, Indianapolis, IN (US); Arieh L Shalhav, Indianapolis, IN (US); Mihir K Biswas, Indianapolis, IN (US)

Correspondence Address: BARNES & THORNBURG, 11 SOUTH MERIDIAN, INDIANAPOLIS, IN, 46204

(21) Appl. No.: 10/380,031

(22) PCT Filed: Sep. 19, 2001
(86) PCT No.: PCT/US01/29359

(51) Int. Cl. A61B 6/00

(52) U.S. Cl. 600/476

(57) ABSTRACT

An apparatus and method employ first, second and third trocars (60) for introduction of equipment into, and removal of equipment from, a body region, an optical imaging system (optical fibers, 72, 76, 84), a source (88) of a relatively inert fluid for expanding the body region to facilitate the introduction of components of the apparatus into the body region to facilitate the introduction of components of apparatus, and an ultrasound apparatus (108, 112, 302, 304) for at least one of visualization and treatment of the body region. A first one of the trocars (60) facilitates passing of the component (optical fibers) of the optical imaging system (optical fibers, 72, 76, 84) into and out of the body region. A second one of the trocars (60) facilitates passing the fluid between the fluid source (88) and the body region. A third one of the trocars (60) facilitates passing the ultrasound visualization and/or treatment apparatus (108, 112, 302, 304) into and out of the body region.

### FIELD OF THE INVENTION

This invention relates to instruments for the conduct of minimally invasive medical procedures which may be conducted with the aid of laparoscopic techniques, and to such procedures themselves. It is disclosed in the context of high-intensity focused ultrasound ablation of kidney tissue, but is believed to be useful in other applications as well.

### BACKGROUND OF THE INVENTION

Several minimally invasive and non-invasive techniques for the treatment of living tissues and organs with ultrasound, including high-intensity, focused ultrasound, sometimes referred to hereinafter as HIFU, are known. There are, for example, the techniques and apparatus described in U.S. Pat. Nos. 4,084,582; 4,207,901; 4,223,560; 4,227,417; 4,248,090; 4,257,271; 4,317,370; 4,325,381; 4,586,512; 4,620,546; 4,658,828; 4,664,121; 4,858,613; 4,951,653; 4,955,365; 5,036,855; 5,054,470; 5,080,102; 5,117,832; 5,149,319; 5,215,680; 5,219,401; 5,247,935; 5,295,484; 5,316,000; 5,391,197; 5,409,006; 5,443,069; 5,470,350; 5,492,126; 5,573,497; 5,601,526; 5,620,479; 5,630,837; 5,643,179; 5,676,692; 5,840,031. The disclosures of these references are hereby incorporated herein by reference. This listing is not intended to be a representation that a thorough search has been made of the relevant art, or that no better references than those listed are available. Nor should any such representation be inferred.

### DISCLOSURE OF THE INVENTION

According to an aspect of the invention, an apparatus and method employ first, second and third devices for introduction of equipment into, and removal of equipment from, a body region, an optical imaging system, a source of a relatively non-reactive fluid for expanding the body region to facilitate the introduction of components of the apparatus into the body region and manipulation of the introduced components of apparatus, and an ultrasound apparatus for at least one of visualization and treatment of the body region. A first of the devices facilitates passing of the component of the optical imaging system into and out of the body region. A second of the devices facilitates passing the fluid between the fluid source and the body region. A third of the devices facilitates passing the ultrasound visualization and/or treatment apparatus into and out of the body region.

Illustratively according to this aspect of the invention, the optical imaging system includes at least one of: a light source; a first optical fiber having a first end adjacent the light source and a second end remote from the light source for coupling light from the light source to the second end of the optical fiber; a second optical fiber for coupling light from a first end of the second optical fiber to a second end of the second optical fiber; and, an optical imaging device coupled to the second end of the second optical fiber.

Further illustratively according to this aspect of the invention, the first device permits sealing introduction of a component of the optical imaging system into, and removal of the component of the optical imaging system from the body region to reduce the likelihood of the escape of the fluid from the body region.

Additionally illustratively according to this aspect of the invention, the first device includes a first device for passing the second end of the first optical fiber and the first end of the second optical fiber into and from the body region.

Illustratively according to this aspect of the invention, the first device includes a first device for sealingly introducing the optical fiber into, and removing the optical fiber from, the body region to reduce the likelihood of the escape of the fluid from the body region.

Further illustratively according to this aspect of the invention, the optical imaging device includes a video camera.

Additionally illustratively according to this aspect of the invention, the optical imaging device includes a surgical monitor.

Illustratively according to this aspect of the invention, the source of relatively non-reactive fluid includes a source of a relatively non-reactive gas or non-reactive mixture of gases.

Further illustratively according to this aspect of the invention, the second device includes a second device for sealingly introducing relatively non-reactive gas or non-reactive mixture of gases into, and removing relatively non-reactive gas or non-reactive mixture of gases from the body region to reduce the likelihood of the escape of the relatively non-reactive gas or non-reactive mixture of gases from the body region.

Additionally illustratively according to this aspect of the invention, the ultrasound apparatus includes an ultrasound transducer for high-intensity focused ultrasound (HIFU) treatment of the body region.

Illustratively according to this aspect of the invention, the third device includes a third device for sealingly introducing the HIFU treatment transducer into, and removing the HIFU treatment transducer from, the body region to reduce the likelihood of the escape of the relatively non-reactive fluid from the body region.

Further illustratively according to this aspect of the invention, the ultrasound apparatus includes an ultrasound transducer for visualization of the body region.

Additionally illustratively according to this aspect of the invention, the third device includes a third device for sealingly introducing the visualization transducer into, and removing the visualization transducer from, the body region to reduce the likelihood of the escape of the relatively non-reactive fluid from the body region.

Illustratively according to this aspect of the invention, the ultrasound visualization and/or treatment apparatus further includes a device for displaying an ultrasound image generated from information received by the transducer. The ultrasound image display device is coupled to the visualization transducer by conductors which extend through the third device.

Further illustratively according to this aspect of the invention, the HIFU treatment and ultrasound visualization transducers are combined into a multi-element ultrasound transducer.

Illustratively according to this aspect of the invention, the ultrasound apparatus for at least one of visualization and treatment of the body region includes multiple ultrasound transducers capable of being driven to provide HIFU treatment of the body region.

Further illustratively according to this aspect of the invention, at least one of the multiple ultrasound transducers capable of being driven to provide HIFU treatment of the body region is also capable of being driven to provide visualization of the body region.

Illustratively according to this aspect of the invention, each of the multiple ultrasound transducers is capable of being driven to provide visualization of the body region.

Illustratively according to this aspect of the invention, the invention further includes introducing species containing at least one of: microcapsule-seeded species; microbubble-seeded species; and, relatively non-reactive gas or relatively non-reactive mixture of gases, and exposing the at least one of: microcapsule-seeded species; microbubble-seeded species; and, relatively non-reactive gas or relatively non-reactive mixture of gases to ultrasound to cause an echogenic field to appear on the ultrasound display device.

Illustratively according to this aspect of the invention, the invention further includes introducing species containing at least one of: microcapsule-seeded species; microbubble-seeded species; and, relatively non-reactive gas or non-reactive mixture of gases, and exposing the at least one of: microcapsule-seeded species; microbubble-seeded species; and, relatively non-reactive gas or non-reactive mixture of gases to ultrasound to cause cavitation of the at least one of: microcapsule-seeded species; microbubble-seeded species; and, relatively non-reactive gas or relatively non-reactive mixture of gases.

Additionally illustratively according to this aspect of the invention, providing an ultrasound apparatus for at least one of visualization and treatment of the body region includes providing a coupling medium between the first ultrasound transducer and tissue to be treated in the body region.

Illustratively according to this aspect of the invention, providing a coupling medium between the first ultrasound transducer and tissue to be treated in the body region includes providing around the first ultrasound transducer a flexible reservoir, providing the coupling medium in the reservoir, and placing the reservoir into contact with tissue to be treated in the body region.

Further illustratively according to this aspect of the invention, the flexible reservoir is constrained to deflect in certain ways when a sufficient volume of the coupling medium is introduced into the flexible reservoir to cause it to deflect.

According to another aspect of the invention, an apparatus and method employ first and second devices for introduction of equipment into, and removal of equipment from, a body region, a source of a relatively non-reactive fluid for expanding the body region to facilitate the introduction of components of the apparatus into the body region and manipulation of the introduced components of apparatus and an ultrasound apparatus for at least one of visualization and treatment of the body region. The fluid passes between the fluid source and the body region through the first device. The ultrasound visualization and/or treatment apparatus passes through the second device.

Illustratively according to this aspect of the invention, the source of a relatively non-reactive fluid for expanding the body region includes a source of a relatively non-reactive gas or non-reactive mixture of gases.

Further illustratively according to this aspect of the invention, the first device includes a first device for sealingly introducing relatively non-reactive fluid into, and removing relatively non-reactive fluid from, the body region.

Additionally illustratively according to this aspect of the invention, the ultrasound apparatus includes a first ultrasound transducer for high-intensity focused ultrasound (HIFU) treatment of the body region.

Illustratively according to this aspect of the invention, the second device includes a second device for sealingly introducing the HIFU treatment transducer into, and removing the HIFU treatment transducer from, the body region.

Further illustratively according to this aspect of the invention, the ultrasound apparatus includes a second ultrasound transducer for visualization of the body region.

Additionally illustratively according to this aspect of the invention, the second device includes a second device for sealingly introducing the visualization transducer into, and removing the visualization transducer from, the body region.

Illustratively according to this aspect of the invention, the ultrasound visualization and/or treatment apparatus further includes a device for displaying an ultrasound image generated from information received by the ultrasound visualization transducer, and conductors for coupling the ultrasound visualization transducer through the second device to the ultrasound display device.

Further illustratively according to this aspect of the invention, the first and second transducers are combined in a multi-element ultrasound transducer.

Additionally illustratively according to this aspect of the invention, the second device includes a second device for sealingly introducing the multi-element ultrasound transducer into, and removing the multi-element ultrasound transducer from, the body region.

Further illustratively according to this aspect of the invention, the ultrasound apparatus for at least one of visualization and treatment of the body region includes multiple ultrasound transducers for providing HIFU treatment of the body region.

Additionally illustratively according to this aspect of the invention, at least one of the multiple ultrasound transducers for providing HIFU treatment of the body region is also capable of being driven to provide visualization of the body region.

Further illustratively according to this aspect of the invention, each of the multiple ultrasound transducers is capable of being driven to provide visualization of the body region.

### BRIEF DESCRIPTION OF THE DRAWINGS

The invention may best be understood by referring to the following detailed description and accompanying drawings which illustrate the invention. In the drawings:

FIG. 1 illustrates a partly block diagrammatic, partly fragmentary perspective view of a procedure according to the present invention;

FIG. 2 illustrates an exploded, fragmentary perspective view of a device useful in the conduct of the procedure illustrated in FIG. 1;

FIG. 3 illustrates a perspective view of another device constructed according to the invention;

FIG. 4 illustrates a perspective view of another device constructed according to the invention;

FIG. 5 illustrates a perspective view of certain components of another device constructed according to the invention;

FIG. 6 illustrates a plan view of the components illustrated in FIG. 5;

FIG. 7 illustrates an elevational view of the components illustrated in FIGS. 5-6; and,

FIG. 8 illustrates an end elevational view of the components illustrated in FIGS. 5-7.

### DETAILED DESCRIPTIONS OF ILLUSTRATIVE EMBODIMENTS

In an illustrated minimally invasive, HIFU-based procedure, the patient 20 is first prepared by the insertion of a guide wire 24 through the urethra 28 and bladder 32 into the ureter 36 of a diseased kidney 40. The guide wire 24 is, of course, radiopaque, so that its progress to the surgical field can be straightforwardly monitored. Then, using the guide wire 24, a urological catheter 44 is inserted along the same path to permit the introduction of fluid species into the surgical site 48. Next, three incisions 50, 52, 54 are made on the abdomen 56 below the diaphragm through trocars 60. The trocars 60 are left in place, as is customary, to permit the sealing of the abdomen 56 when instruments are passed through the seals 64 of the trocars 60 into the abdomen 56 for the conduct of the procedure.

A laparoscope 68 for providing visual observation of the surgical field is passed through one of the trocars 60. The laparoscope 68 is conventionally coupled to a video camera 72 and a light source 76 for illuminating the surgical field and returning images to a surgical monitor 84. The laparoscope provides a pair of fiberoptic ports, one an output port for light from source 76 to the surgical field, and one an input port for the returning image information to video camera 72. A second of the trocars 60 provides, among other things, a passageway for the introduction into the abdomen 56 of a relatively inert gas, such as, for example, carbon dioxide, from a source 88 in order to permit the inflation of the abdomen 56 below the diaphragm. This increases the space inside the abdomen 56 for maneuvering surgical instruments including the laparoscope 68, and provides a clearer view of the surgical field.

The third trocar 60 provides access through the abdominal wall and into the surgical field for a HIFU probe 90 which will be used to ablate the surgical site 48 of a diseased kidney 40, for example, for the virtually bloodless ablation of (a) tumor(s) on the surface of, and/or within, the kidney 40. Should the surgical procedure call for it, additional trocars 60 can, of course, be provided for passing into the body additional HIFU probes 90 to be used in conjunction with each other in an ablation procedure. The presence of the catheter 44 in the kidney 40 also permits the introduction into the surgical field of (an) ablation enhancing medium (media) and other media at (an) appropriate time(s) during the procedure. The same, or a different, medium (media) may also be introduced through the catheter 44 to improve the accuracy of the targeting of the surgical site 48 for ablation and provide feedback to the treating physician of the progress of the treatment. For example, lesions which are not on the surface of the tissue 40 being treated are not easily visible, or in many cases visible at all, in the laparoscopically informed monitor 84.

In order to provide feedback to the treating physician of the progress of treatment of a site 48 not visible on the monitor 84, the ultrasound probe 90 includes an ultrasound visualization capability. (An) additional mechanism(s) may be provided for essentially real-time monitoring of the progress of the treatment. For example, it is known in the ultrasound visualization and therapy arts that there are numerous mechanisms available to promote visualization of the progress of ultrasound treatment within an organ or tissue. These include the introduction of relatively inert gas-containing microcapsule- or microbubble-seeded species, such as sterile saline solution, the introduction of a relatively inert gas, again, such as carbon dioxide, and so on. Any suitable one or ones of these mechanisms can be used to introduce any of such media via the catheter 44 into the kidney 40 being treated. Such materials are known to create bright echogenic bands, strips, fields, and the like on, for example, B-mode ultrasound imaging scans 86. Such phenomena can be used to indicate to the treating physician where the HIFU has been effective. The treating physician continues to expose the tissue 40 under treatment to the HIFU until the material produces a “bloom” or bright echogenic field, band, strip or the like in the ultrasound image 86 of the treatment field. Then the HIFU probe 90 is repositioned to treat the next region which is to be treated according to the treatment regimen. Some of such species, such as relatively inert gas-containing microcapsule-seeded sterile saline solution, microbubble-seeded sterile saline solution, and the like, may also function to enhance the ablation effects of the applied HIFU. For example, some of such species readily produce cavitation, the bursting of bubbles created when the species are exposed to HIFU above certain field strengths and/or for certain lengths of time. The cavitation is known to cause further mechanical alteration of the character of the tissue at the surgical site 48 at a cellular level, enhancing the effects of the HIFU exposure. This ultimately results in reduced treatment times.

This treatment is not limited to kidneys. It is presently believed to be applicable equally readily to the ablation of tissue on the surface of, or in the bulk of, for example, the liver, the pancreas, the urinary bladder 32, the gall bladder, the stomach, the heart, lungs, and so on.

Turning now to the construction of the HIFU probe 90 and related hardware, although the probe 90 was tested by manipulation by the treating physician, it is within the contemplation of the present invention that the probe 90 could be integrated into, or mounted to be manipulated by, a robotic mechanism 92, and controlled, for example, by means of a joystick 94, keypad 96, programmable machine 100, or any other appropriate control mechanism. Any of such mechanisms 92, 94, 96, 100 can incorporate feedback control (illustrated by broken lines), not only of a visual nature, provided via a laparoscope 68, but also of the ultrasound imaging type via probe 90.

The ultrasound image 86 feedback may be not only of the more conventional type described above, but also, may be of a somewhat more highly processed nature, such as that described in, for example, U.S. S. No. 60/200,695, filed Apr. 29, 2000, titled Non-Invasive Tissue Characterization, assigned to the assignee of this application, and hereby incorporated herein by reference. It is contemplated that the feedback could provide the treating physician with highly detailed information on the progress of treatment, such as, for example, when the tissue being treated Teaches a particular temperature, when the character of the tissue at a cellular level changes abruptly, and so on.

The illustrated probe 90 itself is, for example, a modified Sonablate 200 probe available from Focus Surgery, Inc., 3940 Pendleton Way, Indianapolis, Ind., 46226. The Sonablate 200 system is hereby incorporated herein by reference. The probe 90 includes a segmented, curved rectangular elliptical transducer 104 of the general type described in, for example, WO 99/49788. The transducer 104 has a central segment 108 which is used both for visualization and therapy and (an) outer segment(s) 112 which is (are) used for therapy, in accordance with known principles. However, it will immediately be appreciated that other single element or multi-segment transducer configurations, such as ones providing variable focal length, can be used to advantage in other embodiments of the invention. Some of such variable focal length configurations, and driving and receiving systems for them, are described in the prior art incorporated herein by reference.

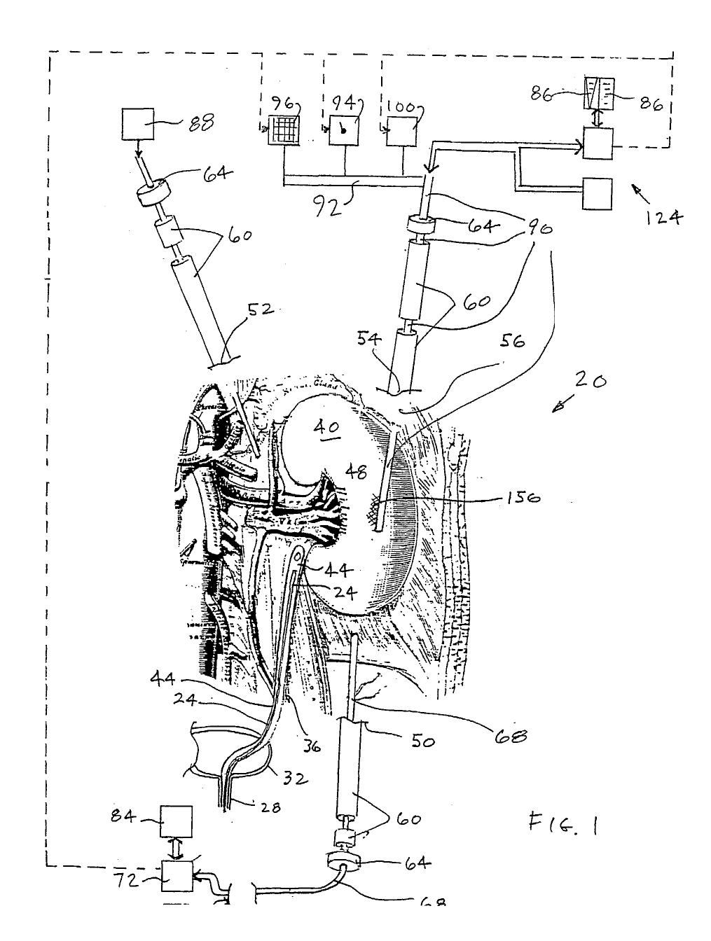

The illustrated transducer 104 has a length of about 3 cm., a width of about 1.3 cm., and a focal length of about 3.5 cm. This is adequate to treat tumors of the kidney 40 to that depth. The HIFU treatment of deeper seated tissue will, of course, require longer focal length treatment transducers. The transducer 104 is mounted in a holder 116 having the same generally rectangular prism-shaped outline as the outer dimensions of the transducer 104 itself. The holder 116 is mounted on the end of a hollow shaft 120 through which the electrical leads to drive the transducer 104 for imaging 86 and therapy can be passed between the transducer 104 and the driver and imaging circuitry, for example, the driver and imaging circuitry of the above-mentioned Sonablate 200 system, in a controller 124 (FIG. 1). The shaft 120 itself can serve as one of the conductors, for example, the ground conductor, for one or more of the ultrasound-generating segment(s) 108, 112 of the transducer 104. The transducer 104/holder 116/shaft 120 assembly is housed in a housing 128 which illustratively is about 50 cm in length and has an outside diameter which is sufficiently small to fit through one of the standard trocar 60 seals 64, for example, an 18 mm seal 64, sufficiently tightly to seal the inside of the abdominal cavity in use. Of course, the dimensions of the illustrated transducer 104, holder 116 and housing 128 given above are for a probe 90 for the treatment of certain kidney 40 tissue. The size, shape and focal length of the probe 90 and transducer 104 will depend to a great extent on the requirements of the tissue or organ which the probe 90 is intended to treat. For example, a liver probe may be required to be somewhat larger and have a longer focal length, and so on.

It should be recalled that it is contemplated that the abdominal cavity will be pressurized with gas during the procedure to increase the work space inside the abdominal cavity. Recalling that a gas will ordinarily be used during the procedure to inflate the abdomen 56, provision must be made for coupling the ultrasound transducer 104 to the tissue being treated. This may be done by providing a cot or condom 132 over the window 136 through the housing 128 through which the ultrasound radiating face 140 of the transducer 104 transmits ultrasound, and filling the housing 128 with an appropriate coupling medium, for example, degassed water and permitting air to escape from the housing 128 as it is being filled. One or more ports may be provided in the housing 128 for filling it with coupling medium and bleeding air from it. The cot 132 may be sealed to the housing 128 longitudinally of the housing 128 on either side of the window 136 by elastomeric O-ring seals 144. This reduces the amount of coupling fluid necessary inside the housing 128 to cause the cot 132 to bulge out sufficiently to bring it into intimate contact with the surface of the tissue 40 to be treated.

To reduce further the amount of coupling fluid necessary inside the housing 128 to cause the cot 132 to bulge out sufficiently to bring it into intimate contact with the surface of the tissue 40 to be treated, a sleeve 148 having an opening 152 corresponding generally in size, shape and orientation to the size, shape and orientation of the window 136, such as, for example, a longitudinally slitted 152 sleeve 148, is placed around the housing 128 in the region of the window 136. The sleeve 148 illustratively is constructed of a thin, sterilizable or sterile disposable material, such as, for example, a resin or light metal. The sleeve 148 slides or snaps around the housing 128 in the region of the ultrasound window 136 after the cot 132 has been placed over the window 136, and either before or after the O-rings 144 have been positioned adjacent the longitudinal ends of the window 136. The sleeve 148 is intended to reduce the bulging of the cot 132 anywhere other than in the immediate vicinity of the window 136. This reduces the amount of coupling fluid necessary to cause the cot 132 to bulge into intimate contact with the tissue 40 by reducing the volume of coupling fluid necessary to cause adequate bulging of the cot 132.

It should also be recalled that ultrasound tissue imaging 86 is deep tissue imaging, not surface imaging. Surface imaging in the illustrated application is provided by the laparoscope 68's vision system 76, 72, 84. It is helpful for both gross and fine positioning of the probe 90, including tissue contact with the cot 132 filled with coupling medium, and for monitoring the progress of treatment. For example, visualization permits the physician to determine when the tissue 40 being treated exhibits surface blanching 156 (FIG. 1). The presence of blanching 156 provides visual feedback to the treating physician that the tissue 40 being treated has received an amount of heat, at least on its surface, to achieve a particular level of ablation. Instead of this surface imaging being provided laparoscopically, this surface imaging could also be provided by means of a light source and video return on the probe 90 itself. The light source and video return on the probe 90 itself might take the form of an LED or other light source provided on the probe 90 adjacent the window 136, and a miniature video image generator of some type also adjacent the window 136, or some other combination of image-generating components.

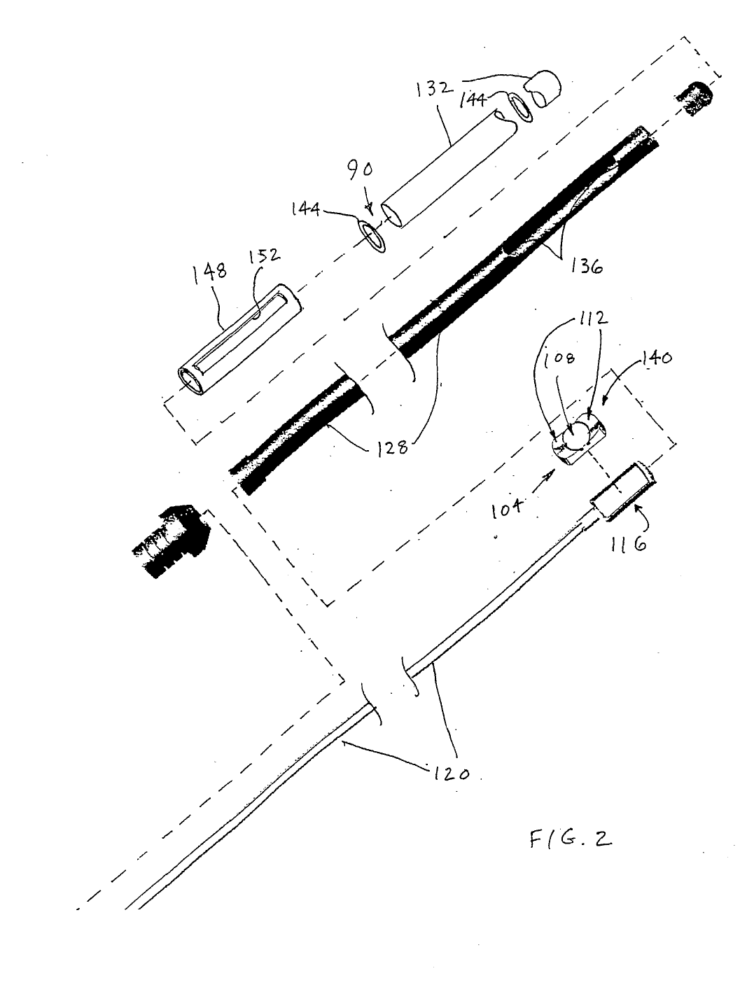

In another embodiment, illustrated in FIG. 3, the probe 180 takes the form of one jaw of a forceps-like clamp 184. The other jaw 188 of the clamp 184 serves with the clamping jaw/probe 180 to capture the tissue 192 to be treated between the two jaws 180, 188. Then, the transducer 104 in the jaw 180 is energized in the same way as discussed above by a driver/receiver/visualization system 124 to treat the tissue 192 with HIFU. In another embodiment, illustrated in FIG. 4, both jaws 280, 288 can take the form of probes so that the tissue 292 to be treated could be treated by both probes 280, 288 or by whichever one of the probes 280, 288 is optimally positioned to treat the tissue 292 to be treated. The ultrasound transducers 104, 104 in the two probe/jaws 280, 288 could have different characteristics, for example, different power handling capabilities or focal lengths, in order to provide a greater number of treatment options to the physician when the probes/jaws 280, 288 are in position to treat the tissue 292.

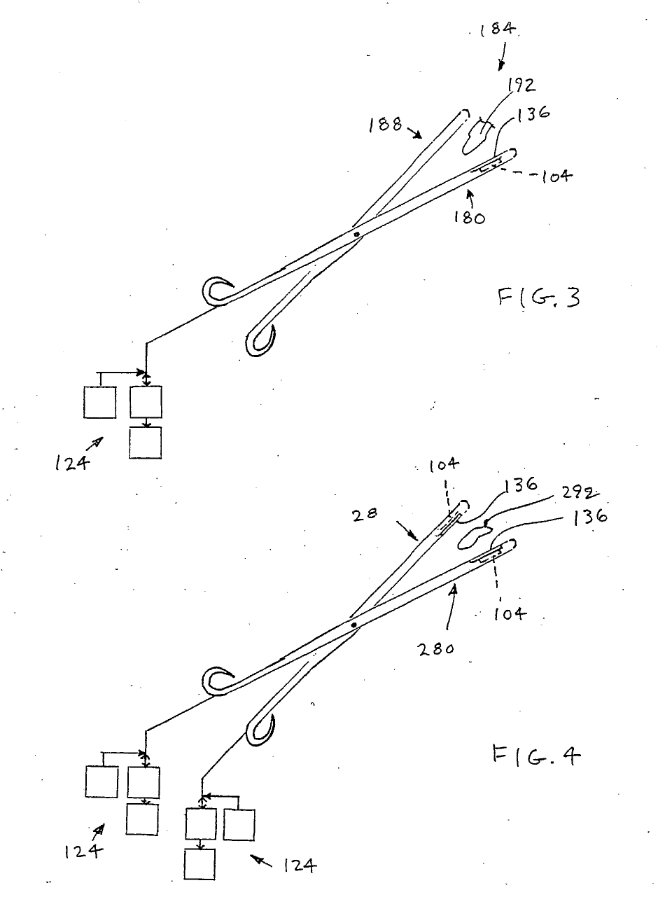

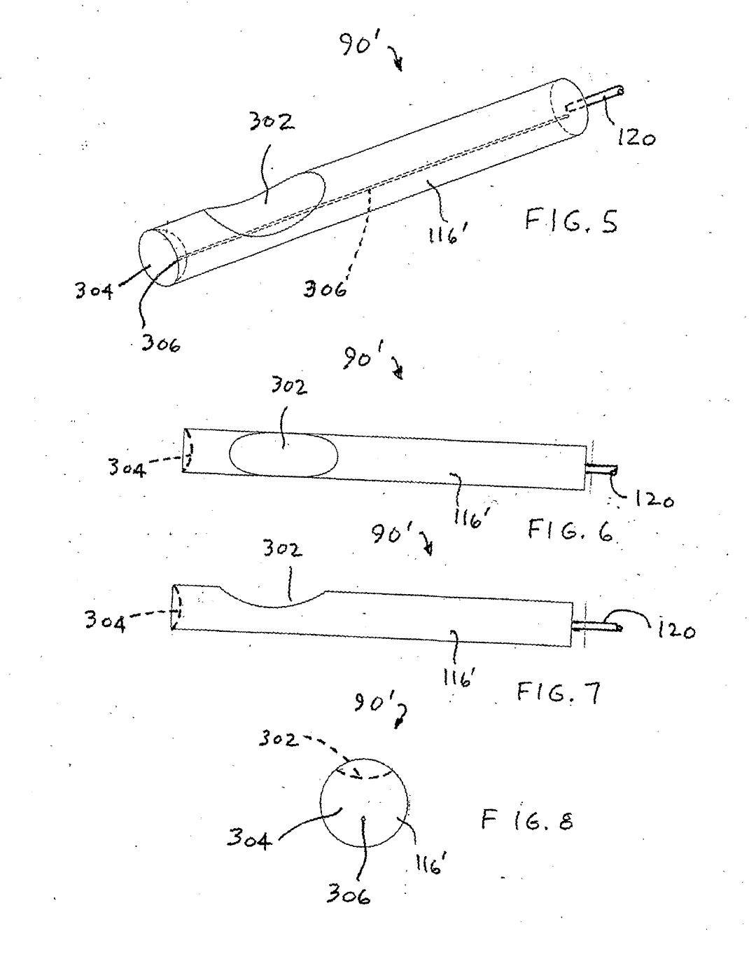

In another embodiment, illustrated in FIGS. 5-8, a probe 90′ includes a holder 116′ for mounting part-spherical visualization and treatment transducers 302, 304 having radii of, for example, 30 mm for transducer 302 and 15 mm for transducer 304. Both of transducers 302, 304 are capable of operation in visualization and HIFU treatment modes. And, of course, either or both of transducers 302, 304 can be a multi-element transducer of any of the known types including transducer 104 illustrated in FIGS. 1-2. In this embodiment, the end cap and the end O-ring seal 144 of the embodiment illustrated in FIGS. 1-2 are omitted to permit the cot 132 to bulge from the end of probe 90′ when the cot 132 is filled with coupling medium, in order that ultrasound may better be coupled from/to the transducer 304 to/from tissue being visualized and/or treated. Holder 116′ also includes its own fiberoptic passageway 306 having a diameter of, for example, 0.5 mm. Passageway 306 extends out to the surface of transducer 304 to provide optical visualization of tissue being treated, which tissue may also be visualized by ultrasound and/or treated by transducer 304. The optical fiber(s) which extend(s) through passageway 306 is (are) coupled to an illumination/optical visualization system of known type, such as the system 72, 76, 84 illustrated and briefly described in connection with the embodiment illustrated in FIGS. 1-2.

1. Apparatus including first, second and third devices for introduction of equipment into, and removal of equipment from, a body region, an optical imaging system, the first device passing a component of the optical imaging system, a source of a relatively non-reactive fluid for expanding the body region to facilitate the introduction of components of the apparatus into the body region and manipulation of the introduced components of apparatus, the second device passing the fluid between the fluid source and the body region, and an ultrasound apparatus for at least one of visualization and treatment of the body region, the third device passing the ultrasound visualization and/or treatment apparatus.

2. The apparatus of claim 1 wherein the optical imaging system includes at least one of: a light source; a first optical fiber having a first end adjacent the light source and a second end remote from the light source for coupling light from the light source to the second end of the optical fiber; a second optical fiber for coupling light from a first end of the second optical fiber to a second end of the second optical fiber; and, an optical imaging device coupled to the second end of the second optical fiber.

3. The apparatus of claim 2 wherein the first device includes a first device for sealingly introducing a component of the optical imaging system into, and removing the component of the optical imaging system from, the body region.

4. The apparatus of claim 2 wherein the first device includes a first device for passing the second end of the first optical fiber and the first end of the second optical fiber into and from the body region.

5. The apparatus of claim 4 wherein the first device includes a first device for sealingly introducing the optical fiber into, and removing the optical fiber from, the body region.

6. The apparatus of claim 2 wherein the optical imaging device includes a video camera.

7. The apparatus of claim 2 wherein the optical imaging device includes a surgical monitor.

8. The apparatus of claim 1 wherein the source of a relatively non-reactive fluid for expanding the body region includes a source of a relatively non-reactive gas or non-reactive mixture of gases.

9. The apparatus of claim 1 wherein the second device includes a second device for sealingly introducing relatively non-reactive fluid into, and removing relatively non-reactive fluid from, the body region.

10. The apparatus of claim 1 wherein the ultrasound apparatus includes a first ultrasound transducer for high-intensity focused ultrasound (HIFU) treatment of the body region.

11. The apparatus of claim 10 wherein the third device includes a third device for sealingly introducing the HIFU treatment transducer into, and removing the HIFU treatment transducer from, the body region.

12. The apparatus of claim 10 wherein the ultrasound apparatus includes a second ultrasound transducer for visualization of the body region.

13. The apparatus of claim 12 wherein the third device includes a third device for sealingly introducing the visualization transducer into, and removing the visualization transducer from, the body region.

14. The apparatus of claim 13 wherein the ultrasound visualization and/or treatment apparatus further includes a device for displaying an ultrasound image generated from information received by the ultrasound visualization transducer, and conductors for coupling the ultrasound visualization transducer to the ultrasound display device, the conductors extending through the third device.

15. The apparatus of claim 12 wherein the first and second transducers are combined in a multi-element ultrasound transducer.

16. The apparatus of claim 15 wherein the third device includes a third device for sealingly introducing the multi-element ultrasound transducer into, and removing the multi-element ultrasound transducer from, the body region.

17. The apparatus of claim 16 wherein the ultrasound visualization and/or treatment apparatus further includes a device for displaying an ultrasound image generated from information received by the ultrasound visualization transducer, and conductors for coupling the ultrasound visualization transducer to the ultrasound display device, the conductors extending through the third device.

18. The apparatus of claim 1 wherein the ultrasound apparatus for at least one of visualization and treatment of the body region includes an ultrasound transducer for visualization of the body region.

19. The apparatus of claim 18 wherein the third device includes a third device for sealingly introducing the visualization transducer into, and removing the visualization transducer from, the body region.

20. The apparatus of claim 19 wherein the ultrasound visualization and/or treatment apparatus further includes a device for displaying an ultrasound image generated from information received by the ultrasound visualization transducer, and conductors extending through the third device for coupling the ultrasound visualization transducer to the ultrasound display device.

21. The apparatus of claim 1 wherein the ultrasound apparatus for at least one of visualization and treatment of the body region includes multiple ultrasound transducers for providing high-intensity focused ultrasound (HIFU) treatment of the body region.

22. The apparatus of claim 21 wherein at least one of the multiple ultrasound transducers for providing HIFU treatment of the body region is also capable of being driven to provide visualization of the body region.

23. The apparatus of claim 22 wherein the ultrasound visualization and/or treatment apparatus further includes a device for displaying an ultrasound image generated from information received by the at least one ultrasound transducer capable of being driven to provide visualization of the body region, and conductors extending through the third device for coupling the at least one ultrasound transducer capable of being driven to provide visualization of the body region to the ultrasound display device.

24. The apparatus of claim 22 wherein each of the multiple ultrasound transducers is capable of being driven to provide visualization of the body region.

25. The apparatus of claim 24 wherein the ultrasound visualization and/or treatment apparatus further includes a device for displaying an ultrasound image generated from information received by each of the multiple ultrasound transducers, and conductors extending through the third device for coupling each of the multiple ultrasound transducers to provide visualization of the body region to the ultrasound display device.

26. A method including providing first, second and third devices for introduction of equipment into, and removal of equipment from, a body region, providing an optical imaging system, passing a component of the optical imaging system through the first device, providing a source of a relatively non-reactive fluid for expanding the body region to facilitate the introduction of components of the apparatus into the body region and manipulation of the introduced components of apparatus, passing the fluid between the fluid source and the body region through the second device, providing an ultrasound apparatus for at least one of visualization and treatment of the body region, and passing the ultrasound visualization and/or treatment apparatus through the third device.

27. The method of claim 26 wherein providing an optical imaging system includes providing at least one of: a light source; a first optical fiber having a first end adjacent the light source and a second end remote from the light source for coupling light from the light source to the second end of the optical fiber; a second optical fiber for coupling light from a first end of the second optical fiber to a second end of the second optical fiber; and, an optical imaging device coupled to the second end of the second optical fiber.

28. The method of claim 27 wherein providing a first device includes providing a first device for sealingly introducing a component of the optical imaging system into, and removing the component of the optical imaging system from the body region.

29. The method of claim 27 wherein providing a first device includes providing a first device for passing the second end of the first optical fiber and the first end of the second optical fiber into and from the body region.

30. The method of claim 29 wherein providing a first device for introduction of the optical fiber into, and removal of the optical fiber from, a body region includes providing a first device for sealingly introducing the optical fiber into, and removing the optical fiber from the body region.

31. The method of claim 27 wherein providing an optical imaging device includes providing a video camera.

32. The method of claim 27 wherein providing an optical imaging device includes providing a surgical monitor.

33. The method of claim 26 wherein providing a source of a relatively non-reactive fluid for expanding the body region includes providing a source of a relatively non-reactive gas or non-reactive mixture of gases.

34. The method of claim 26 wherein providing a second device includes providing a second device for sealingly introducing relatively non-reactive fluid into, and removing relatively non-reactive fluid from the body region to reduce the likelihood of the escape of the relatively non-reactive fluid from the body region.

35. The method of claim 26 wherein providing an ultrasound apparatus for at least one of visualization and treatment of the body region includes providing a first ultrasound transducer for high-intensity focused ultrasound (HIFU) treatment of the body region.

36. The method of claim 35 wherein providing a third device includes providing a third device for sealingly introducing the HIFU treatment transducer into, and removing the HIFU treatment transducer from, the body region.

37. The method of claim 35 wherein providing an ultrasound apparatus for at least one of visualization and treatment of the body region includes providing a second ultrasound transducer for visualization of the body region.

38. The method of claim 37 wherein providing a third device includes providing a third device for sealingly introducing the visualization transducer into, and removing the visualization transducer from, the body region.

39. The method of claim 38 wherein providing an ultrasound visualization and/or treatment apparatus further includes providing a device for displaying an ultrasound image generated from information received by the ultrasound visualization transducer, and coupling the ultrasound visualization transducer to the ultrasound display device through the third device.

40. The method of claim 37 wherein providing first and second transducers includes providing a multi-element ultrasound transducer.

41. The method of claim 40 wherein providing a third device includes providing a third device for sealingly introducing the multi-element ultrasound transducer into, and removing the multi-element ultrasound transducer from, the body region.

42. The method of claim 41 wherein providing an ultrasound visualization and/or treatment apparatus further includes providing a device for displaying an ultrasound image generated from information received by the ultrasound visualization transducer, and coupling the ultrasound visualization transducer to the ultrasound display device through the third device.

43. The method of claim 26 wherein providing an ultrasound apparatus for at least one of visualization and treatment of the body region includes providing an ultrasound transducer for visualization of the body region.

44. The method of claim 43 wherein providing a third device includes providing a third device for sealingly introducing the visualization transducer into, and removing the visualization transducer from, the body region.

45. The method of claim 44 wherein providing an ultrasound visualization and/or treatment apparatus further includes providing a device for displaying an ultrasound image generated from information received by the ultrasound visualization transducer, and coupling the ultrasound visualization transducer through the third device to the ultrasound display device.

46. The method of claim 26 wherein providing ultrasound apparatus for at least one of visualization and treatment of the body region includes providing multiple ultrasound transducers capable of being driven to provide high-intensity focused ultrasound (HIFU) treatment of the body region.

47. The method of claim 46 wherein providing multiple ultrasound transducers capable of being driven to provide HIFU treatment of the body region further includes providing at least one of the multiple ultrasound transducers capable of being driven to provide visualization of the body region.

48. The method of claim 47 wherein providing ultrasound visualization and/or treatment apparatus further includes providing a device for displaying an ultrasound image generated from information received by the at least one ultrasound transducer capable of being driven to provide visualization of the body region, and coupling the at least one ultrasound transducer capable of being driven to provide visualization of the body region through the third device to the ultrasound display device.

49. The method of claim 47 wherein providing multiple ultrasound transducers includes providing multiple ultrasound transducers, each capable of being driven to provide visualization of the body region.

50. The method of claim 49 wherein providing ultrasound visualization and/or treatment apparatus further includes providing a device for displaying an ultrasound image generated from information received by each of the multiple ultrasound transducers, and coupling each of the multiple ultrasound transducers through the third device to provide visualization of the body region to the ultrasound display device.

51. The method of claim 45 further including introducing at least one of: microcapsule-seeded species; microbubble-seeded species; and, relatively non-reactive gas or relatively non-reactive mixture of gases, and exposing the at least one of: microcapsule-seeded species; microbubble-seeded species; and, relatively non-reactive gas or relatively non-reactive mixture of gases to ultrasound to cause an echogenic field to appear on the ultrasound display device.

52. The method of claim 35 further including introducing at least one of: microcapsule-seeded species; microbubble-seeded species; and, relatively non-reactive gas, and exposing the at least one of: microcapsule-seeded species; microbubble-seeded species; and, relatively non-reactive gas or relatively non-reactive mixture of gases to ultrasound to cause cavitation of the at least one of: microcapsule-seeded species; microbubble-seeded species; and, relatively non-reactive gas or relatively non-reactive mixture of gases.

53. The method of claim 35 wherein providing an ultrasound apparatus for at least one of visualization and treatment of the body region includes providing a coupling medium between the first ultrasound transducer and tissue to be treated in the body region.

54. The method of claim 53 wherein providing a coupling medium between the first ultrasound transducer and tissue to be treated in the body region includes providing around the first ultrasound transducer a flexible reservoir, providing the coupling medium in the reservoir, and placing the reservoir into contact with tissue to be treated in the body region.

55. The method of claim 54 further including constraining the flexible reservoir to deflect in certain ways when a sufficient volume of the coupling medium is introduced into the flexible reservoir to cause it to deflect.

56. Apparatus including first and second devices for introduction of equipment into, and removal of equipment from, a body region, a source of a relatively non-reactive fluid for expanding the body region to facilitate the introduction of components of the apparatus into the body region and manipulation of the introduced components of apparatus, the first device passing the fluid between the fluid source and the body region, and an ultrasound apparatus for at least one of visualization and treatment of the body region, the second device passing the ultrasound visualization and/or treatment apparatus.

57. The apparatus of claim 56 wherein the source of a relatively non-reactive fluid for expanding the body region includes a source of a relatively non-reactive gas or non-reactive mixture of gases.

58. The apparatus of claim 56 wherein the first device includes a first device for sealingly introducing relatively non-reactive fluid into, and removing relatively non-reactive fluid from, the body region.

59. The apparatus of claim 56 wherein the ultrasound apparatus includes a first ultrasound transducer for high-intensity focused ultrasound (HIFU) treatment of the body region.

60. The apparatus of claim 59 wherein the second device includes a second device for sealingly introducing the HIFU treatment transducer into, and removing the HIFU treatment transducer from, the body region.

61. The apparatus of claim 59 wherein the ultrasound apparatus includes a second ultrasound transducer for visualization of the body region.

62. The apparatus of claim 61 wherein the second device includes a second device for sealingly introducing the visualization transducer into, and removing the visualization transducer from, the body region.

63. The apparatus of claim 62 wherein the ultrasound visualization and/or treatment apparatus further includes a device for displaying an ultrasound image generated from information received by the ultrasound visualization transducer, and conductors for coupling the ultrasound visualization transducer to the ultrasound display device, the conductors extending through the second device.

64. The apparatus of claim 61 wherein the first and second transducers are combined in a multi-element ultrasound transducer.

65. The apparatus of claim 64 wherein the second device includes a second device for sealingly introducing the multi-element ultrasound transducer into, and removing the multi-element ultrasound transducer from, the body region.

66. The apparatus of claim 65 wherein the ultrasound visualization and/or treatment apparatus further includes a device for displaying an ultrasound image generated from information received by the ultrasound visualization transducer, and conductors for coupling the ultrasound visualization transducer to the ultrasound display device, the conductors extending through the second device.

67. The apparatus of claim 56 wherein the ultrasound apparatus for at least one of visualization and treatment of the body region includes an ultrasound transducer for visualization of the body region.

68. The apparatus of claim 67 wherein the second device includes a second device for sealingly introducing the visualization transducer into, and removing the visualization transducer from, the body region.

69. The apparatus of claim 68 wherein the ultrasound visualization and/or treatment apparatus further includes a device for displaying an ultrasound image generated from information received by the ultrasound visualization transducer, and conductors extending through the second device for coupling the ultrasound visualization transducer to the ultrasound display device.

70. The apparatus of claim 56 wherein the ultrasound apparatus for at least one of visualization and treatment of the body region includes multiple ultrasound transducers for providing high-intensity focused ultrasound (HIFU) treatment of the body region.

71. The apparatus of claim 70 wherein at least one of the multiple ultrasound transducers for providing HIFU treatment of the body region is also capable of being driven to provide visualization of the body region.

72. The apparatus of claim 71 wherein the ultrasound visualization and/or treatment apparatus further includes a device for displaying an ultrasound image generated from information received by the at least one ultrasound transducer capable of being driven to provide visualization of the body region, and conductors extending through the second device for coupling the at least one ultrasound transducer capable of being driven to provide visualization of the body region to the ultrasound display device.

73. The apparatus of claim 71 wherein each of the multiple ultrasound transducers is capable of being driven to provide visualization of the body region.

74. The apparatus of claim 73 wherein the ultrasound visualization and/or treatment apparatus further includes a device for displaying an ultrasound image generated from information received by each of the multiple ultrasound transducers, and conductors extending through the second device for coupling each of the multiple ultrasound transducers to provide visualization of the body region to the ultrasound display device.

75. A method including providing first and second devices for introduction of equipment into, and removal of equipment from, a body region, providing a source of a relatively non-reactive fluid for expanding the body region to facilitate the introduction of components of the apparatus into the body region and manipulation of the introduced components of apparatus, passing the fluid between the fluid source and the body region through the first device, providing an ultrasound apparatus for at least one of visualization and treatment of the body region, and passing the ultrasound visualization and/or treatment apparatus through the second device.

76. The method of claim 75 wherein providing a source of a relatively non-reactive fluid for expanding the body region includes providing a source of a relatively non-reactive gas or non-reactive mixture of gases.

77. The method of claim 75 wherein providing a first device includes providing a first device for sealingly introducing relatively non-reactive fluid into, and removing relatively non-reactive fluid from the body region to reduce the likelihood of the escape of the relatively non-reactive fluid from the body region.

78. The method of claim 75 wherein providing an ultrasound apparatus for at least one of visualization and treatment of the body region includes providing a first ultrasound transducer for high-intensity focused ultrasound (HIFU) treatment of the body region.

79. The method of claim 78 wherein providing a second device includes providing a second device for sealingly introducing the HIFU treatment transducer into, and removing the HIFU treatment transducer from, the body region.

80. The method of claim 78 wherein providing an ultrasound apparatus for at least one of visualization and treatment of the body region includes providing a second ultrasound transducer for visualization of the body region.

81. The method of claim 80 wherein providing a second device includes providing a second device for sealingly introducing the visualization transducer into, and removing the visualization transducer from, the body region.

82. The method of claim 81 wherein providing an ultrasound visualization and/or treatment apparatus further includes providing a device for displaying an ultrasound image generated from information received by the ultrasound visualization transducer, and coupling the ultrasound visualization transducer to the ultrasound display device through the second device.

83. The method of claim 80 wherein providing first and second transducers includes providing a multi-element ultrasound transducer.

84. The method of claim 83 wherein providing a second device includes providing a second device for sealingly introducing the multi-element ultrasound transducer into, and removing the multi-element ultrasound transducer from, the body region.

85. The method of claim 84 wherein providing an ultrasound visualization and/or treatment apparatus further includes providing a device for displaying an ultrasound image generated from information received by the ultrasound visualization transducer, and coupling the ultrasound visualization transducer to the ultrasound display device through the second device.

86. The method of claim 75 wherein providing an ultrasound apparatus for at least one of visualization and treatment of the body region includes providing an ultrasound transducer for visualization of the body region.

87. The method of claim 86 wherein providing a second device includes providing a second device for sealingly introducing the visualization transducer into, and removing the visualization transducer from, the body region.

88. The method of claim 87 wherein providing an ultrasound visualization and/or treatment apparatus further includes providing a device for displaying an ultrasound image generated from information received by the ultrasound visualization transducer, and coupling the ultrasound visualization transducer through the second device to the ultrasound display device.

89. The method of claim 75 wherein providing ultrasound apparatus for at least one of visualization and treatment of the body region includes providing multiple ultrasound transducers capable of being driven to provide high-intensity focused ultrasound (HIFU) treatment of the body region.

90. The method of claim 89 wherein providing multiple ultrasound transducers capable of being driven to provide HIFU treatment of the body region further includes providing at least one of the multiple ultrasound transducers capable of being driven to provide visualization of the body region.

91. The method of claim 90 wherein providing ultrasound visualization and/or treatment apparatus further includes providing a device for displaying an ultrasound image generated from information received by the at least one ultrasound transducer capable of being driven to provide visualization of the body region, and coupling the at least one ultrasound transducer capable of being driven to provide visualization of the body region through the second device to the ultrasound display device.

92. The method of claim 90 wherein providing multiple ultrasound transducers includes providing multiple ultrasound transducers, each capable of being driven to provide visualization of the body region.

93. The method of claim 92 wherein providing ultrasound visualization and/or treatment apparatus further includes providing a device for displaying an ultrasound image generated from information received by each of the multiple ultrasound transducers, and coupling each of the multiple ultrasound transducers through the second device to provide visualization of the body region to the ultrasound display device.

94. The method of claim 88 further including introducing at least one of: microcapsule-seeded species; microbubble-seeded species; and, relatively non-reactive gas or relatively non-reactive mixture of gases, and exposing the at least one of: microcapsule-seeded species; microbubble-seeded species; and, relatively non-reactive gas or relatively non-reactive mixture of gases to ultrasound to cause an echogenic field to appear on the ultrasound display device.

95. The method of claim 78 further including introducing at least one of: microcapsule-seeded species; microbubble-seeded species; and, relatively non-reactive gas, and exposing the at least one of: microcapsule-seeded species; microbubble-seeded species; and, relatively non-reactive gas or relatively non-reactive mixture of gases to ultrasound to cause cavitation of the at least one of: microcapsule-seeded species; microbubble-seeded species; and, relatively non-reactive gas or relatively non-reactive mixture of gases.

96. The method of claim 78 wherein providing an ultrasound apparatus for at least one of visualization and treatment of the body region includes providing a coupling medium between the first ultrasound transducer and tissue to be treated in the body region.

97. The method of claim 96 wherein providing a coupling medium between the first ultrasound transducer and tissue to be treated in the body region includes providing around the first ultrasound transducer a flexible reservoir, providing the coupling medium in the reservoir, and placing the reservoir into contact with tissue to be treated in the body region.

98. The method of claim 97 further including constraining the flexible reservoir to deflect in certain ways when a sufficient volume of the coupling medium is introduced into the flexible reservoir to cause it to deflect.
